SEQUENCE LISTING (1 sequence)

SEQ ID NO: 1 - protein, 662 aa, Homo sapiens
MGLYRIRVSTGASLYAGSNNQVQLWLVGQHGEAALGKRLWPARGKETELKVEVPEYLGPLLFVKLRKRHLLKDDAWFCNWISVQGPGAGDEVRFPCYRWVEGNGVLSLPEGTGRTVGEDPQGLFQKHREEELEERRKLYRWGNWKDGLILNMAGAKLYDLPVDERFLEDKRVDFEVSLAKGLADLAIKDSLNVLTCWKDLDDFNRIFWCGQSKLAERVRDSWKEDALFGYQFLNGANPVVLRRSAHLPARLVFPPGMEELQAQLEKELEGGTLFEADFSLLDGIKANVILCSQQHLAAPLVMLKLQPDGKLLPMVIQLQLPRTGSPPPPLFLPTDPPMAWLLAKCWVRSSDFQLHELQSHLLRGHLMAEVIVVATMRCLPSIHPIFKLIIPHLRYTLEINVRARTGLVSDMGIFDQIMSTGGGGHVQLLKQAGAFLTYSSFCPPDDLADRGLLGVKSSFYAQDALRLWEIIYRYVEGIVSLHYKTDVAVKDDPELQTWCREITEIGLQGAQDRGFPVSLQARDQVCHFVTMCIFTCTGQHASVHLGQLDWYSWVPNAPCTMRLPPPTTKDATLETVMATLPNFHQASLQMSITWQLGRRQPVMVAVGQHEEEYFSGPEPKAVLKKFREELAALDKEIEIRNAKLDMPYEYLRPSVVENSVAI

(19) United States
(12) Patent Application Publication
(10) Pub. No.: US 2025/0064899 A1
(43) Pub. Date: Feb. 27, 2025

(54) USE OF THE 15-LIPOXYGENASE FOR THE TREATMENT OF LYMPHEDEMA

(71) Applicants: INSERM (INSTITUT NATIONAL DE LA SANTÉ ET DE LA RECHERCHE MÉDICALE), Paris (FR); UNIVERSITÉ TOULOUSE III - PAUL SABATIER, Toulouse (FR)

(72) Inventor: Barbara GARMY-SUSINI, Toulouse (FR)

(21) Appl. No.: 18/728,953

(22) PCT Filed: Feb. 13, 2023
(86) PCT No.: PCT/EP2023/053482

§ 371 Date: Jul. 15, 2024

(30) Foreign Application Priority Data
Feb. 14, 2022 (EP) 22305165.7

(51) Int. Cl. A61K 38/44; A61K 48/00; A61P 7/00; C12N 15/86

(52) U.S. Cl. CPC A61K 38/44; A61P 7/00; C12N 15/86; A61K 48/00; C12N 2740/15043; C12N 2750/14143

(57) ABSTRACT

Lymphedema is characterized by the accumulation of protein-rich interstitial fluid, lipids and a significant inflammatory cell infiltrate in the limb. It causes a significant morbidity and is a common disabling disease affecting more than 250 million people worldwide, however there is no curative treatment for lymphedema. Here, the inventors found that dermolipectomies from patient with lymphedema exhibit inflamed gene expression profile compared to normal arm on same patient. After lipidomic analysis, the inventors identified severe decrease in arachidonic acid-derived lipid mediators generated by the 15-lipoxygenase (15-LOX) in lymphedematous arms. Using a mouse model of lymphedema, they reproduced the etiology of the human pathology including the loss of specialized pro-resolving lipid mediators that play essential roles in resolution of inflammation. This was associated with a lack of regulatory T cells (Treg) recruitment in the injured limb adipose tissue. Importantly, the inventors identified the lymphatic endothelial 15-LOX was responsible for the chemoattraction and transendothelial migration of Tregs. These results were confirmed by an aggravation of lymphedema and deterioration of the lymphatic network in an original transgenic mouse model in which ALOX15 gene is selectively deleted in the lymphatic system. Importantly, this was reversed by the injection of 15-LOX expressing lentivectors. These results provide evidence that lymphatic lipoxygenase may represent a novel therapeutic target for lymphedema by serving as a mediator of Treg invasion into lymphedematous adipose tissue.

### FIELD OF THE INVENTION

The present invention is in the field of medicine, in particular treatment of lymphedema.

### BACKGROUND OF THE INVENTION

The lymphatic system provides a conduit for reabsorption of the interstitial fluid that escapes from the arteriovenous circulation. Additional vital lymphatic functions include gastrointestinal lipid absorption and the trafficking of immune cells¹. In the face of heritable defects or acquired lymphatic vascular insults, the resultant lymphatic dysfunction induces loss of normal immune responses, or the development of lymphatic vascular insufficiency, known as Lymphedema (LD)². LD is characterized by the accumulation of protein-rich interstitial fluid, a significant inflammatory cell infiltrate, and dysregulated regional immune responses. Subsequent adipose tissue deposition and fibrosis, at the site of lymphatic malfunction, promotes progressive anatomic distortion and loss of function and chronic inflammation².

In response to injury, the acute inflammation is timely orchestrated protective program that is critical for the tissue repair and restoration of homeostasis³. It is divided into 2 phases: initiation and resolution. The initiation phase is marked by tissue edema resulting from increased blood flow and permeability of the microvasculature. Polymorphonuclear neutrophils (PMN) migrate to the site of injury in response to chemical signals including proinflammatory lipid mediators (e.g. leukotriene B₄ [LTB₄]) and chemokines. The resolution phase is already being enacted at this early point as the influx of PMN is halted at a level appropriate for the insult and is accompanied by their timely apoptosis⁴. Then, monocytes infiltrate the tissue where they differentiate into macrophages to perform the resolution phase. They respond to damage-associated molecular signature present in the injured area for tissue repair and regeneration, allowing for the return to homeostasis⁴⁻⁸. Regulatory T (Treg) cell responses represent critical arms of the inflammation resolution response as they improve the apoptotic cell clearance (efferocytosis) and thus increase the resolution⁹.

A defect in clearance can lead to exacerbated inflammation, impeding tissue repair. In that context, adaptive immune cells play critical roles in the host response to resolution of inflammation and in tissue repair leading to chronic inflammation and thus^{10,11} resultant in tissue damage, excessive fibrosis, and loss of function, as observed in lymphedema.

The resolution of vascular inflammation is an important driver of vessel wall remodeling and functional recovery. Pro-resolving lipid mediators, derived from polyunsaturated fatty acids, orchestrate key cellular processes favoring resolution and a return to immune homeostasis. The discovery of their presence in lymphatic vessels has thus generated great interest in their role in LD-induced inflammation. Importantly, studies have demonstrated that T cells are key regulators of inflammation in LD¹². They revealed that recruitment of Tregs to inflamed LD tissues suppresses the T_{H}1/T_{H}2 immune response and limits tissue fibrosis, leading to improved lymphatic function. Human lipoxygenases (LOXs) catalyze the stereoselective dioxygenation of polyunsaturated fatty acids (arachidonic acid (AA), DHA and EPA)¹³. In particular, the 15-LOX enzyme is constitutively expressed in immune- and endothelial cells, where it contributes to immune modulation. Indeed, the resolution of inflammation is impaired in 15-LOX-deficient mice and is accompanied with impaired wound healing response with increased post-inflammatory fibrosis¹⁴. The 15-LOX catalyzes the oxygenation of AA thus generating 15-Hydroxyeicosatetraenoic acids (15-HETE) metabolites. The 15-HETE mediator displays chemotactic activities, notably impacting neutrophil trafficking¹⁵, and stimulates VEGFA synthesis, thus indirectly affecting endothelial activation¹⁶. It differs from the 12-HETE that is produced by 12-LOX and has been described to promote circular chemorepellent defects on the lymphatic monolayer when synthetized by tumor cells¹⁷.

Pro-resolving lipid mediators are mostly locally synthesized by immune cells. However, they are also produced in vascular tissues including endothelial cells. These lipids have direct effects on endothelial-leukocyte interactions, and play a protective role following injury¹⁸. Hence, they are considered as potential vascular therapeutics, but also as candidate biomarkers in vascular disease.

Lymphedema is a chronic pathological condition associated with inflammation. Exploratory studies support the utility of targeted anti-inflammatory therapy with ketoprofen in patients with lymphedema¹⁹. Conversely, mouse model of obesity promotes adipose tissue inflammation, impairs lymphatic vascular function and exacerbates lymphedema. Mechanistically, increased infiltration of T helper 2 (Th2) cells impairs both lymphangiogenic response of capillaries and collecting vessel function, in part through the production of Th2 cytokines IL-4 and IL-13²⁰.

Additionally, accumulating CD11b⁺ macrophages produce high levels of nitric oxide and disrupt lymphatic endothelial NO signaling, leading to decreased collecting lymphatic contractility and function²¹. Thus, several inflammatory and immune cell subsets are detrimental to normal LEC function and they contribute to obesity-associated lymphatic function impairment. On the other hand, regulatory T cells (Tregs) that curb the inflammation, have been shown to promote lymphatic vessel regeneration and repair in mouse model of tail lymphedema²².

### SUMMARY OF THE INVENTION

The present invention is defined by the claims. In particular, the present invention relates to the use of the 15-Lipoxygenase (15-LOX) for the treatment of lymphedema.

### DETAILED DESCRIPTION OF THE INVENTION

Lymphedema is characterized by the accumulation of protein-rich interstitial fluid, lipids and a significant inflammatory cell infiltrate in the limb. It causes a significant morbidity and is a common disabling disease affecting more than 250 million people worldwide, however there is no curative treatment for lymphedema. Here, the inventors found that dermolipectomies from patient with lymphedema exhibit inflamed gene expression profile compared to normal arm on same patient. After lipidomic analysis, the inventors identified severe decrease in arachidonic acid-derived lipid mediators generated by the 15-lipoxygenase (15-LOX) in lymphedematous arms. Using a mouse model of lymphedema, they reproduced the etiology of the human pathology including the loss of specialized pro-resolving lipid mediators that play essential roles in resolution of inflammation. This was associated with a lack of regulatory T cells (Treg) recruitment in the injured limb adipose tissue. Importantly, the inventors identified the lymphatic endothelial 15-LOX was responsible for the chemoattraction and transendothelial migration of Tregs. These results were confirmed by an aggravation of lymphedema and deterioration of the lymphatic network in an original transgenic mouse model in which ALOX15 gene is selectively deleted in the lymphatic system (Prox1CreERT2;ALOX15^{fl/fl}). Importantly, this was reversed by the injection of 15-LOX expressing lentivectors. These results provide evidence that lymphatic lipoxygenase may represent a novel therapeutic target for lymphedema by serving as a mediator of Treg invasion into lymphedematous adipose tissue.

Accordingly, the first object of the present invention relates to a method of treating lymphedema in a patient in need thereof comprising administering to the patient a therapeutically effective amount of i) a 15-LOX polypeptide or ii) a polynucleotide encoding for a 15-LOX polypeptide.

As used herein, the term “lymphedema” has its general meaning in the art and refers to a disorder characterized by a strong tissue swelling due to an increased fluid retention in the tissue, a local accumulation of adipose tissue and an impairment of immune function due to reduced lymphatic drainage. The term includes “primary lymphedema” and “secondary lymphedema”. Primary lymphedema is a lymphatic system malformation characterized by swelling of an extremity that can be associated with other lymphatic effusions, due to an underlying developmental anomaly of the lymphatic system (abnormal lymphangiogenesis). It can be hereditary or not and be congenital or late onset. In some embodiments, lymphedema is found as secondary disorder which may result from lymph node dissection or injury of lymphatic vessels. Such secondary lymphedema may also accompany, e.g., lymph node dissection and injury in connection with surgery, radiation therapy, tumor disease and treatments thereof, musculoskeletal injuries like fractures, tendon releases and joint replacements, neurological conditions like muscle paresis, vascular injuries/surgeries, integumentary injuries, coagulation disorders such as deep vein thrombosis, scar tissue formation, tamoxifen treatment, filariasis, infection, lipedema or cellulitis. The term “treating lymphedema” encompasses both preventive and curative treatment of lymphedema.

In particular, the polypeptide or the polynucleotide of the present invention is particularly suitable for improving invasion of Treg cells in the tissues and thus resolution of inflammation.

As used herein, the term “regulatory T cells” or “Treg cells” refers to cells that suppress, inhibit or prevent T cells activity. Typically, Treg cells have the following phenotype at rest CD4+CD25+FoxP3+.

As used herein, the term “15-LOX” refers to the polyunsaturated fatty acid lipoxygenase 15-Lipoxygenase. An exemplary amino acid sequence for 15-LOX is represented by SEQ ID NO:1. The lipoxygenase domain ranges from the amino acid residue at position 115 to the amino acid 662.

>sp|P16050|LOX15_HUMAN Polyunsaturated fatty acid
lipoxygenase ALOX15 OS = Homo sapiens OX = 9606
GN = ALOX15 PE = 1 SV = 3. The lipoxygenase
domain is underlined.
SEQ ID NO: 1
MGLYRIRVSTGASLYAGSNNQVQLWLVGQHGEAALGKRLWPARGKETEL
KVEVPEYLGPLLFVKLRKRHLLKDDAWFCNWISVQGPGAGDEVRFPCYR
WVEGNGVLSLPEGTGRTVGEDPQGLFQKHREEELEERRKLYRWGNWKDG
LILNMAGAKLYDLPVDERFLEDKRVDFEVSLAKGLADLAIKDSLNVLTC
WKDLDDFNRIFWCGQSKLAERVRDSWKEDALFGYQFLNGANPVVLRRSA
HLPARLVFPPGMEELQAQLEKELEGGTLFEADFSLLDGIKANVILCSQQ
HLAAPLVMLKLQPDGKLLPMVIQLQLPRTGSPPPPLFLPTDPPMAWLLA
KCWVRSSDFQLHELQSHLLRGHLMAEVIVVATMRCLPSIHPIFKLIIPH
LRYTLEINVRARTGLVSDMGIFDQIMSTGGGGHVQLLKQAGAFLTYSSF
CPPDDLADRGLLGVKSSFYAQDALRLWEIIYRYVEGIVSLHYKTDVAVK
DDPELQTWCREITEIGLQGAQDRGFPVSLQARDQVCHFVTMCIFTCTGQ
HASVHLGQLDWYSWVPNAPCTMRLPPPTTKDATLETVMATLPNFHQASL
QMSITWQLGRRQPVMVAVGQHEEEYFSGPEPKAVLKKFREELAALDKEI
EIRNAKLDMPYEYLRPSVVENSVAI

As used herein, the term “polypeptide” has its general meaning in the art and refers to a polymer of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

As used herein, the term “15-LOX polypeptide” refers to a polypeptide that comprises the lipoxygenase domain of 15-LOX.

In some embodiments, the 15-LOX polypeptide of the present invention comprises an amino acid sequence having at least 90% of identity with the amino acid sequence that ranges from the amino acid residue at position 115 to the amino acid residue at position 662 in SEQ ID NO:1.

As used herein, the “percent identity” between two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “polynucleotide” refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some embodiments, the polynucleotide comprises an mRNA. In other aspect, the mRNA is a synthetic mRNA. In some embodiments, the synthetic mRNA comprises at least one unnatural nucleobase. In some embodiments, all nucleobases of a certain class have been replaced with unnatural nucleobases (e.g., all uridines in a polynucleotide disclosed herein can be replaced with an unnatural nucleobase, e.g., 5-methoxyuridine). In some embodiments, the polynucleotide (e.g., a synthetic RNA or a synthetic DNA) comprises only natural nucleobases, i.e., A, C, T and G in the case of a synthetic DNA, or A, C, T, and U in the case of a synthetic RNA.

In some embodiments, the polynucleotide of the present invention is a messenger RNA (mRNA).

In some embodiments, the polynucleotide is inserted in a vector, such a viral vector.

As used herein, the term “viral vector” refers to a virion or virus particle that functions as a nucleic acid delivery vehicle and which comprises a vector genome packaged within the virion or virus particle. Typically, the vector is a viral vector which is an adeno-associated virus (AAV), a retroviral vector, bovine papilloma virus, an adenovirus vector, a vaccinia virus, or a polyoma virus.

In some embodiments, the viral vector is a AAV vector.

As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences.

In some embodiments, the viral vector is a retroviral vector.

As used herein, the term “retroviral vector” refers to a vector containing structural and functional genetic elements that are primarily derived from a retrovirus.

In some embodiments, the retroviral vector of the present invention derives from a retrovirus selected from the group consisting of alpharetroviruses (e.g., avian leukosis virus), betaretroviruses (e.g., mouse mammary tumor virus), gammaretroviruses (e.g., murine leukemia virus), deltaretroviruses (e.g., bovine leukemia virus), epsilonretroviruses (e.g., Walley dermal sarcoma virus), lentiviruses (e.g., HIV-1, HIV-2) and spumaviruses (e.g., human spumavirus).

In some embodiments, the retroviral vector of the present invention is a replication deficient retroviral virus particle, which can transfer a foreign imported RNA of a gene instead of the retroviral mRNA.

In some embodiments, the retroviral vector of the present invention is a lentiviral vector.

As used herein, the term “lentiviral vector” refers to a vector containing structural and functional genetic elements that are primarily derived from a lentivirus. In some embodiments, the lentiviral vector of the present invention is selected from the group consisting of HIV-1, HIV-2, SIV, FIV, EIAV, BIV, VISNA and CAEV vectors. In some embodiments, the lentiviral vector is a HIV-1 vector.

The structure and composition of the vector genome used to prepare the retroviral vectors of the present invention are in accordance with those described in the art. Especially, minimum retroviral gene delivery vectors can be prepared from a vector genome, which only contains, apart from the recombinant nucleic acid molecule of the present invention, the sequences of the retroviral genome which are non-coding regions of said genome, necessary to provide recognition signals for DNA or RNA synthesis and processing. In some embodiment, the retroviral vector genome comprises all the elements necessary for the nucleic import and the correct expression of the polynucleotide of interest (i.e. the transgene). As examples of elements that can be inserted in the retroviral genome of the retroviral vector of the present invention are at least one (preferably two) long terminal repeats (LTR), such as a LTR5′ and a LTR3′, a psi sequence involved in the retroviral genome encapsidation, and optionally at least one DNA flap comprising a cPPT and a CTS domains. In some embodiments of the present invention, the LTR, preferably the LTR3′, is deleted for the promoter and the enhancer of U3 and is replaced by a minimal promoter allowing transcription during vector production while an internal promoter is added to allow expression of the transgene. In particular, the vector is a Self-INactivating (SIN) vector that contains a non-functional or modified 3′ Long Terminal Repeat (LTR) sequence. This sequence is copied to the 5′ end of the vector genome during integration, resulting in the inactivation of promoter activity by both LTRs. Hence, a vector genome may be a replacement vector in which all the viral coding sequences between the 2 long terminal repeats (LTRs) have been replaced by the recombinant nucleic acid molecule of the present invention.

In some embodiments, the retroviral vector genome is devoid of functional gag, pol and/or env retroviral genes. By “functional” it is meant a gene that is correctly transcribed, and/or correctly expressed. Thus, the retroviral vector genome of the present invention in this embodiment contains at least one of the gag, pol and env genes that is either not transcribed or incompletely transcribed; the expression “incompletely transcribed” refers to the alteration in the transcripts gag, gag-pro or gag-pro-pol, one of these or several of these being not transcribed. In some embodiments, the retroviral genome is devoid of gag, pol and/or env retroviral genes.

In some embodiments the retroviral vector genome is also devoid of the coding sequences for Vif-, Vpr-, Vpu- and Nef-accessory genes (for HIV-1 retroviral vectors), or of their complete or functional genes.

Typically, the retroviral vector of the present invention is non replicative i.e., the vector and retroviral vector genome are not able to form new particles budding from the infected host cell. This may be achieved by the absence in the retroviral genome of the gag, pol or env genes, as indicated in the above paragraph; this can also be achieved by deleting other viral coding sequence(s) and/or cis-acting genetic elements needed for particles formation.

The retroviral vectors of the present invention can be produced by any well-known method in the art including by transfection (s) transient (s), in stable cell lines and/or by means of helper virus. Use of stable cell lines may also be preferred for the production of the vectors (Greene, M. R. et al. Transduction of Human CD34+Repopulating Cells with a Self-Inactivating Lentiviral Vector for SCID-X1 Produced at Clinical Scale by a Stable Cell Line. Hum. Gene Ther. Methods 23, 297-308 (2012).) For instance, the retroviral vector of the present invention is obtainable by a transcomplementation system (vector/packaging system) by transfecting in vitro a permissive cell (such as 293T cells) with a plasmid containing the retroviral vector genome of the present invention, and at least one other plasmid providing, in trans, the gag, pol and env sequences encoding the polypeptides GAG, POL and the envelope protein(s), or for a portion of these polypeptides sufficient to enable formation of retroviral particles. As an example, permissive cells are transfected with a) transcomplementation plasmid, lacking packaging signal psi and, the plasmid is optionally deleted of accessory genes vif, nef, vpu and/or vpr, b) a second plasmid (envelope expression plasmid or pseudotyping env plasmid) comprising a gene encoding an envelope protein(s) and c) a plasmid vector comprising a recombinant genome retroviral, optionally deleted from the promoter region of the 3′LTR or U3 enhancer sequence of the 3′ LTR, including, between the LTR sequences 5′ and 3′ retroviral, a psi encapsidation sequence, a nuclear export element (preferably RRE element of HIV or other retroviruses equivalent), comprising the nucleic acid molecule of the present invention and optionally a promoter and/or a nuclear import sequence (cPPT sequence eg CTS) of the RNA. Advantageously, the three plasmids used do not contain homologous sequence sufficient for recombination. Nucleic acids encoding gag, pol and env cDNA can be advantageously prepared according to conventional techniques, from viral gene sequences available in the prior art and databases. The trans-complementation plasmid provides a nucleic acid encoding the proteins retroviral gag and pol. These proteins are derived from a lentivirus, and most preferably, from HIV-1. The plasmid is devoid of encapsidation sequence, sequence coding for an envelope, accessory genes, and advantageously also lacks retroviral LTRs. Therefore, the sequences coding for gag and pol proteins are advantageously placed under the control of a heterologous promoter, eg cellular, viral, etc . . . , which can be constitutive or regulated, weak or strong. It is preferably a plasmid containing a sequence transcomplémentant Δpsi-CMV-gag-pol-PolyA. This plasmid allows the expression of all the proteins necessary for the formation of empty virions, except the envelope glycoproteins. The plasmid transcomplementation may advantageously comprise the TAT and REV genes. Plasmid transcomplementation is advantageously devoid of vif, vpr, vpu and/or nef accessory genes. It is understood that the gag and pol genes and genes TAT and REV can also be carried by different plasmids, possibly separated. In this case, several plasmids are used transcomplementation, each encoding one or more of said proteins. The promoters used in the plasmid transcomplementation, the envelope plasmid and the plasmid vector respectively to promote the expression of gag and pol of the coat protein, the mRNA of the vector genome and the transgene are promoters identical or different, chosen advantageously from ubiquitous promoters or specific, for example, from viral promoters CMV, TK, RSV LTR promoter and the RNA polymerase III promoter such as U6 or H1 or promoters of helper viruses encoding env, gag and pol (i.e. adenoviral, baculoviral, herpes viruses). For the production of the retroviral vector of the present invention, the plasmids described above can be introduced into competent cells and viruses produced are harvested. The cells used may be any cell competent, particularly eukaryotic cells, in particular mammalian, eg human or animal. They can be somatic or embryonic stem or differentiated. Typically the cells include 293T cells, fibroblast cells, hepatocytes, muscle cells (skeletal, cardiac, smooth, blood vessel, etc.), nerve cells (neurons, glial cells, astrocytes) of epithelial cells, renal, ocular etc. It may also include, insect, plant cells, yeast, or prokaryotic cells. It can also be cells transformed by the SV40 T antigen. The genes gag, pol and env encoded in plasmids or helper viruses can be introduced into cells by any method known in the art, suitable for cell type considered. Usually, the cells and the vector system are contacted in a suitable device (plate, dish, tube, pouch, etc . . . ), for a period of time sufficient to allow the transfer of the vector system or the plasmid in the cells. Typically, the vector system or the plasmid is introduced into the cells by calcium phosphate precipitation, electroporation, transduction or by using one of transfection-facilitating compounds, such as lipids, polymers, liposomes and peptides, etc. The calcium phosphate precipitation is preferred. The cells are cultured in any suitable medium such as RPMI, DMEM, a specific medium to a culture in the absence of fetal calf serum, etc. Once transfected the retroviral vectors of the present invention may be purified from the supernatant of the cells. Purification of the retroviral vector to enhance the concentration can be accomplished by any suitable method, such as by density gradient purification (e.g., cesium chloride (CsCl)) or by chromatography techniques (e.g., column or batch chromatography). For example, the vector of the present invention can be subjected to two or three CsCl density gradient purification steps. The vector, is desirably purified from cells infected using a method that comprises lysing cells infected with adenovirus, applying the lysate to a chromatography resin, eluting the adenovirus from the chromatography resin, and collecting a fraction containing the retroviral vector of the present invention.

In some embodiments, the vector of the present invention includes “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In some embodiments, the polypeptide or polynucleotide of the present invention can be conjugated to at least one other molecule. Typically, said molecule is selected from the group consisting of polynucleotides, polypeptides, lipids, lectins, carbohydrates, vitamins, cofactors, and drugs. In some embodiments, the polypeptide or polynucleotide of the present invention is formulated using one or more lipid-based structures that include but are not limited to liposomes, lipoplexes, or lipid nanoparticles (Paunovska, Kalina, David Loughrey, and James E. Dahlman. “Drug delivery systems for RNA therapeutics.” Nature Reviews Genetics (2022): 1-16). Liposomes are artificially-prepared vesicles which can primarily be composed of a lipid bilayer and can be used as a delivery vehicle for the administration of pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations. As a non-limiting example, liposomes such as synthetic membrane vesicles are prepared by the methods, apparatus and devices described in US Patent Publication No. US20130177638, US20130177637, US20130177636, US20130177635, US20130177634, US20130177633, US20130183375, US20130183373 and US20130183372. In some embodiments, the liposomes are formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (as described in US20100324120) and liposomes which can deliver small molecule drugs such as, but not limited to, DOXIL® from Janssen Biotech, Inc. (Horsham, Pa.). The polypeptide of polynucleotide of the present invention can be encapsulated by the liposome and/or it can be contained in an aqueous core which can then be encapsulated by the liposome (see International Pub. Nos. WO2012031046, WO2012031043, WO2012030901 and WO2012006378 and US Patent Publication No. US20130189351, US20130195969 and US20130202684). In some embodiments, the polynucleotide of the present invention is formulated with stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104).

By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved.

Concomitantly, the Inventors herein identified the crucial role of the lymphatic endothelial production of 15-HETE, a 15-LOX metabolite, as key regulator in lymphedema.

As used herein, the term “15-HETE” refers to 15-hydroxyeicosatetraenoic acid. The 15-LOX catalyzes the oxygenation of AA thus generating 15-hydroxyeicosatetraenoic acids (15-HETE) metabolites. The 15-HETE mediator displays chemotactic activities, notably impacting neutrophil trafficking and stimulates VEGFA synthesis. An exemplary representation of the 15-HETE chemical structure is shown below:

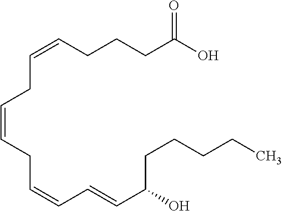 [SMILES: CCCCC[C@H](O)/C=C/C=C\C/C=C\C/C=C\CCCC(=O)O]

The results depicted in the present patent application favor the hypothesis that Treg recruitment initiated by the lymphatic endothelial 15-HETE is a central event in the resolution of inflammation in lymphedema. Importantly, 15-HETE did not cause lymphatic damages as observed with 12-HETE and thus could represent a better therapy for lymphedema.

Accordingly, in another aspect, the present invention also relates to a method of treating lymphedema in a patient in need thereof comprising administering to the patient a therapeutically effective amount of a 15-LOX metabolite, wherein the 15-LOX metabolite is 15-HETE.

Typically the active ingredient of the present invention (i.e. the polypeptide or polynucleotide) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In some embodiments, the pharmaceutical composition comprises i) a 15-LOX polypeptide, ii) a polynucleotide encoding for a 15-LOX polypeptide, iii) 15-HETE and/or iv) a 15-HETE derivative.

As used herein, the term “15-HETE derivative” refers to a compound derived from another after transformation of the latter. As example, the 15-HETE derivative may differ from 15-HETE by one or more atoms or functional groups.

The invention will be further illustrated by the following figures and examples. However, these examples and figures should not be interpreted in any way as limiting the scope of the present invention.

### FIGURES

FIG. 1: Lymphatic endothelial ALOX15 controls lymphedema in vivo. Quantification of the limb diameter in LD from mice treated with intradermal injections of ALOX15 lentivectors (***p<0.001).

FIG. 2: 15-HETE inhibits lymphedema. Quantification of the limb diameter in LD from mice treated with intradermal injections of 300 ng 15-HETE (*p<0.01).

FIG. 3: 15-HETE inhibits dermal lymph backflow in lymphedema. Left panel, Lymphography of the limb from 15-HETE-treated mice. Right panel, Quantification of lymphatic branch point in 15-HETE-treated mice. (*p<0.05).

### EXAMPLE 1

### Main Methods

### Human Tissue Specimen.

In total, 17 lipodermectomy specimens were collected. Samples were obtained from archival paraffin blocks of lymphedema from patients treated at the Rangueil hospital, Toulouse, France between 2015 and 2016. Samples were selected as coded specimens under a protocol approved by the INSERM Institutional Review Board (DC-2008-452) and Research State Department (Ministere de la recherche, ARS, CPP2, authorization AC-2008-452). When it was possible, some control arm tissue samples were collected for esthetical purpose.

Isolated adipocytes were obtained from arm subcutaneous adipose tissues from healthy adult women undergoing elective surgical procedures of fat removal for aesthetic purpose at the Rangueil hospital, Toulouse, France. Informed consent, concerning the use of the AT samples for research only was asked before plastic surgery. Briefly, after collagenase (Sigma) and dispase (Gibco) digestion, the upper phase containing mature adipocytes was collected and washed three times in ECBM medium (Promocell, Heidelberg, Germany) containing 0.5% of free fatty acid bovine albumin (Sigma, France). Mature adipocytes were maintained in a cell culture cassette (Clinicell25, Laboratoires MABIO International, Tourcoing, France) fully filled with ECBM/BSA medium. After 24 h incubation at 37° C. in a humidified 5% CO₂ cell incubator and centrifugation at 50 g during 2 min, the lower phase was recovered, filtered through a 0.22 μm filter and aliquots were frozen until use.

### Mice Models

All studies received local ethics review board approval and were performed in accordance with the guidelines of the European Convention for the Protection of Vertebrate Animals used for experimentation and according to the INSERM IACUC (France) guidelines for laboratory animal husbandry. All animal experiments were approved by the local branch Inserm Rangueil-Purpan of the Midi-Pyrenees ethics committee, France. Animals from different cages in the same experimental group were selected to assure randomization.

Mice were housed in individually ventilated cages in a temperature and light regulated room in a SPF facility and received food and water ad libitum. Female C57BL/6J (6 weeks old) were obtained from Janvier (Le Genest Saint Isle, France). Mouse model of lymphedema was performed as previously described (Morfoisse ATVB 2008).

### Results

### Human Lymphedema Tissues Exhibit an Inflammatory Profile.

To broadly identify gene expression signatures associated to secondary lymphedema, we performed bulk-RNA sequencing on dermolipectomies from four patients and a differential expression analysis (DEseq) followed by a protein-coding RNA profiling (data not shown). Analysis of the data identified 211 mRNAs that are up- (182 genes) or down-regulated (29 genes) in arm with lymphedema (LD) compared with control arm (CTL) (data not shown).

Overall, hierarchical clustering analysis indicates that inflammatory and lipid metabolism-associated genes are specifically regulated in LD (data not shown). In particular, we observed a strong upregulation of PTX3 that is expressed in response to inflamed endothelium²⁴, amphiregulin (AREG) that stimulates VEGFC expression²⁵, an IL6, the major proinflammatory cytokine (data not shown).

Surprisingly, the majority of genes involved in lymphangiogenesis were upregulated including VEGFC and its receptor Flt4 (data not shown). This probably led to the increase density of anarchic, leaky and dysfunctional lymphatics observed in lymphedematous limb when performing patient lymphangiography. We observed an upregulation of genes involved in matrix remodeling including A Disintegrin and Metalloproteinase with Thrombospondin Motifs (ADAMTS) and Tenascin C (TNC) (data not shown). In the opposite, we observed a downregulation of genes associated with in heparane sulfate (HS2ST1, MAMDC2, FGFR2) (data not shown).

To confirm the inflammatory status in lymphedema, flow cytometry analysis of dermoplipectomies was performed in lymphedematous arm compared to the normal arm on the same patients. Among the immune cell populations, we found an increase in lymphocytes recruitment compared to macrophages (data not shown). In particular, T cells (CD4-positive) were upregulated to B lymphocytes (data not shown).

Lymphedema adipose tissue exhibits a decrease in inflammation resolution lipid mediators. LD promotes a massive accumulation of adipose tissue in the limb that is in part responsible of the stasis of interstitial fluids leading to inflammatory cells accumulation. To understand whether lipid composition could be associated with changes in immune cells recruitment, we performed lipidomic analysis of dermolipectomies from lymphedematous arm compared to non-injured arm (not shown). Differential analysis of arachidonic acid (AA), docosahexaenoic acid (DHA), and eicosapentaenoic acid (EPA) were performed on skin and adipose tissue (data not shown). We observed an overall decrease in lipid-derived mediators in both tissues. In particular, we found that arachidonic acid (AA)-derived lipid mediators were strongly reduced in lymphedema, in particular pro-resolvin molecules generated by the 15-Lipoxygenase (15-LOX) (data not shown). We observed a decrease in 15-Hydroxyeicosatetraenoic acid (15-HETE), Lipoxin A4 an B4 (LXA4 and LXB4), the major AA-derivatives (data not shown). In contrast, we did not find any difference in lipids generated by the DHA metabolism (data not shown). This was not explained by the overall expression mRNA or protein expression of 15-LOX in lymphedematous tissues (data not shown). This discordance might be associated with the poor amount of analyzed tissue (μg) compared to the patient's explant (up to 2.5 kg). However, the number of 15-LOX positive cells were decrease in lymphedema tissue samples (data not shown). More importantly, we observed for the first time that 15-LOX was expressed in the lymphatic endothelium (data not shown).

### 15-LOX Activity is Reduced in Murine Model of Lymphedema

To understand the molecular mechanisms underlying the inflammatory process in lymphedema, we performed a time course analysis of the adipose tissue in a mouse model of lymphedema (not shown). In this model based on a mastectomy associated with a brachial and axillary lymphadenectomy, mice exhibited an increased dermal back flow associated with a significant swelling of the limb (data not shown). In early stages of LD (2 weeks post surgery), no significant difference in lipid content was observed (data not shown). In contrast, in acquired lymphedema (8 weeks post-surgery), we found similar pattern as those observed in human tissue samples (data not shown). In particular, we observed a decrease in AA-derived lipid mediators generated by 15-LOX (15-HETE) whereas no difference was found in lipids generated by DHA metabolism (17-HDOHE) (data not shown). After eight weeks of LD, we observed that changes in pro-resolvin content was associated with a decrease in 15-LOX mRNA and protein expression (data not shown). Importantly, the downregulation of 15-LOX was also observed in the lymphatic endothelium (data not shown).

### 15-LOX Promotes Tregs Recruitment in Lymphedematous Tissues

To evaluate the role of 15-LOX in LD, we used the PD146176 pharmacological inhibitor. In presence of 15-LOX inhibitor, LD swelling is increased (data not shown) and is associated with dermal fibrosis (data not shown) followed by a strong dermal backflow (data not shown). As we previously shown that Tcell populations were recruited during LD development, we next performed flow cytometry analysis. We observed in the presence of 15-LOX inhibitor an increase in CD4+ Tcells during LD associated with a decrease in Treg recruitment (data not shown). This was confirmed by a decrease of Tregs (Foxp3 positive cells) immunodetection in lymphedematous tissues (data not shown).

### 15-LOX Controls Tcells Adhesion to LECs

To investigate whereas lymphatic endothelial 15-LOX could control Tcells trafficking, we knocked down 15-LOX in primary cultures of HDLEC using siRNA (data not shown). Despite a poor knock down (30%), we observed a significant downregulation of chemoattractive cytokines CCL21 in LEC-ALOX15KD (data not shown). We did not find any regulation of SPHK1 and the Sphingosine-1-Phosphate Receptor 1 (SlPRl) (data not shown), whereas SPHK2 that maintains the endothelial integrity was upregulated (data not shown)²⁶. The 15-LOX knock down significantly reduced lymphotoxin beta receptor (LTBR) that plays a crucial role in lymphatic transendothelial Treg migration (data not shown)²⁷. We also observed a reduction of Claudin 5 adhesion molecule expression that was associated with an increase of the lymphatic endothelial inflamed status as shown by iNOS overexpression (data not shown).

To decipher the role of 15-Lox lipid derived mediators, we studied the effect of 15-HETE on the cultured LEC. We identified a direct effect of 15 HETE on the lymphatic endothelium. It promoted Tcells adhesion to the lymphatic monolayer (data not shown), had no effect on LEC proliferation (data not shown), but stimulated their migration (data not shown).

### Selective Lymphatic Endothelial 15-LOX Initiates Inflammation Resolution in Lymphedema.

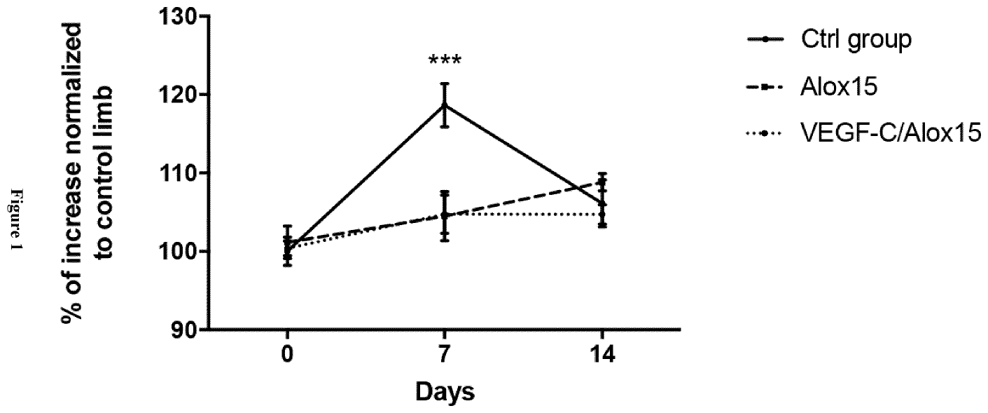

To study selectively the role of lymphatic endothelial 15-LOX, we generated the Prox1CreERT2;Alox15^{fl/fl} conditional knock-out mice in which ALOX15 gene is selectively invalidated into the lymphatic system after tamoxifen induction (data not shown). We observed a significant aggravation of lymphedema in homozygous and heterozygous mice (data not shown). This was not associated with a reduction of lymphangiogenesis in the lymphedematous limb (FIGS. 6c and d). To study the effect of 15-LOX as potential treatment for lymphedema, we generated a lentivector overexpressing the enzyme. Three intradermal injection of 15-LOX lentivector were performed at the time of surgery. In this rescue experiment, the LD development was completely abrogated using lentivector confirming the crucial role of 15-LOX in LD (FIG. 1).

### Discussion

Secondary lymphedema represents a major complication of cancer treatment. It is a multifactorial pathology characterized by a lymphatic endothelial dysfunction, an accumulation of fluid and adipose tissue, a strong dermal fibrosis and a chronic inflammation in the arm or in the leg. Despite many comorbidities associated with lymphedema, it was surprising to observe such reproducible gene expression profile when comparing the lymphedematous arm to the control arm in each patient. Half of these genes were involved in the activation of inflammation, which is in line with previous studies showing the crucial role of inflammation in lymphedema development. As lymphedema develops months, sometimes years after cancer treatment, it is obvious to speculate that an invisible chronic inflammation is developing during months before physical signs such as skin fibrosis occur. To date, it is well accepted that anti-inflammatory drugs are the best candidates to treat some symptoms of lymphedema. Unresolved inflammation is central to the pathophysiology of common vascular diseases such as atherosclerosis, aneurysm, and deep vein thrombosis 5. The resolution of vascular inflammation is an important driver of vessel wall remodeling and functional recovery in these clinical settings. In that context, pro-resolving lipid mediators derived from omega-3 polyunsaturated fatty acids orchestrate key cellular processes driving resolution and a return to homeostasis. The identification of their effects on the lymphatic vessels thus arouses great interest in their properties in lymphedema.

Also, recent study has shown an elevated Leukotriene B4 (LTB4) synthesis in patients with lymphedema²⁸. LTB4 was harmful to lymphatic repair at the concentrations observed in established disease suggesting that LTB4 is a promising drug target for the treatment of acquired lymphedema. We confirmed this hypothesis in a lipidomic analysis of lymphedema tissues that showed significant downregulation of AA-derived lipid mediators including LTB4 in lymphedema.

Pro-resolving lipid mediators are locally synthesized in vascular tissues. They have direct effects on vascular cells-leukocytes interactions, and they play a protective role in the injury response. Therefore, they are considered as potential vascular therapeutics, as well as candidate biomarkers in vascular disease. Here, we investigated the molecular and cellular mechanisms of resolution in the lymphatic vasculature, to improve tools for clinical measurement, and to better define the potential for “resolution therapeutics” in lymphedema patients. Among the different enzyme that generate lipid mediators, we identified that the 15-LOX was expressed on both immune and lymphatic endothelial cells suggesting an autrocrine role on the lymphatic function. As previously described by Krejaschki and colleagues, no deleterious effect of 15-HETE is observed on the lymphatic endothelium compared to the 12-HETE that generates holes in the lymphatic monolayer¹⁷. In our study, we did not find any regulation of 12-HETE in lymphedema. However, Krejaschki's study was restricted to a paracrine effect of 12-HETE produced by tumor cells during metastatic process. Here, we identified for the first time the presence of 15-LOX in the lymphatic endothelial cells. Importantly, the expression of 15-LOX was correlated with a decrease in Treg, in particular Foxp3+CD4+ subset infiltration into the lymphedematous adipose tissue.

Treg cells accumulate in a variety of nonlymphoid tissues to exert both anti-inflammatory and homeostatic functions²⁹. Recently, a novel subset of Treg has been identified in visceral adipose tissue^{30,31,32}. They exhibit a distinct transcriptome from those of lymphoid- and nonlymphoid-tissues^{29,32,33} and their accumulation is dependent on IL-33³⁰. Here, we found that 15-HETE induces CCL21 synthesis by lymphatic endothelial cells to stimulate Treg chemoattraction.

Importantly, using ALOX15^{LECKO} mice, we identified the crucial role of the lymphatic endothelial production of 15-HETE as key regulator in lymphedema.

Taken together, these complementary results favor the hypothesis that Treg recruitment initiated by the lymphatic endothelial 15-HETE is a central event in the resolution of inflammation in lymphedema. Importantly, 15-HETE did not cause lymphatic damages as observed with 12-HETE¹⁷ and thus could represent a potential therapy for lymphedema without promoting any metastatic processes.

### EXAMPLE 2

15-HETE Protects from Lymphedema

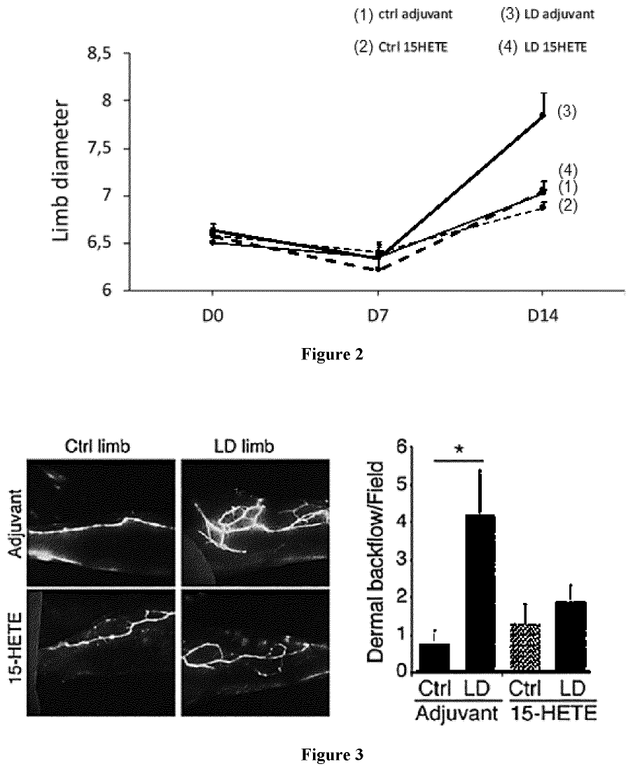

To evaluate the effect of 15-HETE on lymphedema, mice were injected with 300 ng intradermally in the limb at the time of surgery (FIG. 2). Compared with control mice injected with 15-HETE adjuvant (Ethanol), mice did not develop lymphedema when treated with 15-HETE. This was associated with a strong reduction of dermal lymph backflow (FIG. 3), a specific hallmark of lymphedema.

### REFERENCES

Throughout this application, various references describe the state of the art to which this invention pertains. The disclosures of these references are hereby incorporated by reference into the present disclosure.
1 Alitalo, K., Tammela, T. & Petrova, T. V. Lymphangiogenesis in development and human disease. Nature 438, 946-953 (2005).
2 Mortimer, P. S. & Rockson, S. G. New developments in clinical aspects of lymphatic disease. The Journal of clinical investigation 124, 915-921, doi:10.1172/JCI71608 (2014).
3 Serhan, C. N. et al. Resolution of inflammation: state of the art, definitions and terms. FASEB journal: official publication of the Federation of American Societies for Experimental Biology 21, 325-332, doi:10.1096/fj.06-7227rev (2007).
4 Buckley, C. D., Gilroy, D. W. & Serhan, C. N. Proresolving lipid mediators and mechanisms in the resolution of acute inflammation. Immunity 40, 315-327, doi:10.1016/j.immuni.2014.02.009 (2014).
5 Serhan, C. N. Pro-resolving lipid mediators are leads for resolution physiology. Nature 510, 92-101, doi:10.1038/nature13479 (2014).
6 Gimbrone, M. A., Jr. & Garcia-Cardena, G. Endothelial Cell Dysfunction and the Pathobiology of Atherosclerosis. Circulation research 118, 620-636, doi:10.1161/CIRCRESAHA.115.306301 (2016).
7 Kolaczkowska, E. & Kubes, P. Neutrophil recruitment and function in health and inflammation. Nat Rev Immunol 13, 159-175, doi:10.1038/nri3399 (2013).
8 Poon, I. K., Lucas, C. D., Rossi, A. G. & Ravichandran, K. S. Apoptotic cell clearance: basic biology and therapeutic potential. Nat Rev Immunol 14, 166-180, doi:10.1038/nri3607 (2014).
9 Proto, J. D. et al. Regulatory T Cells Promote Macrophage Efferocytosis during Inflammation Resolution. Immunity 49, 666-677 e666, doi:10.1016/j.immuni.2018.07.015 (2018).
10 Fullerton, J. N. & Gilroy, D. W. Resolution of inflammation: a new therapeutic frontier. Nat Rev Drug Discov 15, 551-567, doi:10.1038/nrd.2016.39 (2016).
11 Kupper, T. S. & Fuhlbrigge, R. C. Immune surveillance in the skin: mechanisms and clinical consequences. Nat Rev Immunol 4, 211-222, doi:10.1038/nril310 (2004).
12 Garcia Nores, G. D. et al. CD4(+) T cells are activated in regional lymph nodes and migrate to skin to initiate lymphedema. Nature communications 9, 1970, doi:10.1038/s41467-018-04418-y (2018).
13 Dobrian, A. D. et al. Functional and pathological roles of the 12- and 15-lipoxygenases. Prog Lipid Res 50, 115-131, doi:10.1016/j.plipres.2010.10.005 (2011).
14 Gronert, K. et al. A role for the mouse 12/15-lipoxygenase pathway in promoting epithelial wound healing and host defense. J Biol Chem 280, 15267-15278, doi:10.1074/jbc.M410638200 (2005).
15 Takata, S. et al. Remodeling of neutrophil phospholipids with 15(S)-hydroxyeicosatetraenoic acid inhibits leukotriene B4-induced neutrophil migration across endothelium. The Journal of clinical investigation 93, 499-508, doi:10.1172/JCI116999 (1994).
16 Chen, L. et al. The 15-LO-1/15-HETE system promotes angiogenesis by upregulating VEGF in ischemic brains. Neurol Res 39, 795-802, doi:10.1080/01616412.2017.1321710 (2017).
17 Kerjaschki, D. et al. Lipoxygenase mediates invasion of intrametastatic lymphatic vessels and propagates lymph node metastasis of human mammary carcinoma xenografts in mouse. The Journal of clinical investigation 121, 2000-2012, doi:10.1172/JCI44751 (2011).
18 Karki, P. & Birukov, K. G. Lipid mediators in the regulation of endothelial barriers. Tissue Barriers 6, e1385573, doi:10.1080/21688370.2017.1385573 (2018).
19 Rockson, S. G. et al. Pilot studies demonstrate the potential benefits of antiinflammatory therapy in human lymphedema. JCI insight 3, doi:10.1172/jci.insight.123775 (2018).
20 Ly, C. L., Nores, G. D. G., Kataru, R. P. & Mehrara, B. J. T helper 2 differentiation is necessary for development of lymphedema. Transl Res 206, 57-70, doi:10.1016/j.trsl.2018.12.003 (2019).
21 Liao, S. et al. Impaired lymphatic contraction associated with immunosuppression. Proceedings of the National Academy of Sciences of the United States of America 108, 18784-18789, doi:10.1073/pnas.1116152108 (2011).
22 Gousopoulos, E. et al. Regulatory T cell transfer ameliorates lymphedema and promotes lymphatic vessel function. JCI insight 1, e89081, doi:10.1172/jci.insight.89081 (2016).
23 Jiang, X., Nicolls, M. R., Tian, W. & Rockson, S. G. Lymphatic Dysfunction, Leukotrienes, and Lymphedema. Annu Rev Physiol 80, 49-70, doi:10.1146/annurev-physiol-022516-034008 (2018).
24 Carrizzo, A. et al. Pentraxin 3 Induces Vascular Endothelial Dysfunction Through a P-selectin/Matrix Metalloproteinase-1 Pathway. Circulation 131, 1495-1505; discussion 1505, doi:10.1161/CIRCULATIONAHA.114.014822 (2015).
25 Huang, Y. W. et al. Amphiregulin Promotes Vascular Endothelial Growth Factor-C Expression and Lymphangiogenesis through STAT3 Activation in Human Chondrosarcoma Cells. Cell Physiol Biochem 52, 1-15, doi:10.33594/000000001 (2019).
26 Dimasi, D. P., Pitson, S. M. & Bonder, C. S. Examining the Role of Sphingosine Kinase-2 in the Regulation of Endothelial Cell Barrier Integrity. Microcirculation 23, 248-265, doi:10.1111/micc.12271 (2016).
27 Piao, W. et al. LTbetaR Signaling Controls Lymphatic Migration of Immune Cells. Cells 10, doi:10.3390/cells10040747 (2021).
28 Tian, W. et al. Leukotriene B4 antagonism ameliorates experimental lymphedema. Sci Transl Med 9, doi:10.1126/scitranslmed.aal3920 (2017).
29 Panduro, M., Benoist, C. & Mathis, D. Tissue Tregs. Annu Rev Immunol 34, 609-633, doi:10.1146/annurev-immunol-032712-095948 (2016).
30 Li, C. et al. TCR Transgenic Mice Reveal Stepwise, Multi-site Acquisition of the Distinctive Fat-Treg Phenotype. Cell 174, 285-299 e212, doi:10.1016/j.cell.2018.05.004 (2018).
31 Burzyn, D., Benoist, C. & Mathis, D. Regulatory T cells in nonlymphoid tissues. Nature immunology 14, 1007-1013, doi:10.1038/ni.2683 (2013).
32 Feuerer, M., Shen, Y., Littman, D. R., Benoist, C. & Mathis, D. How punctual ablation of regulatory T cells unleashes an autoimmune lesion within the pancreatic islets. Immunity 31, 654-664, doi:10.1016/j.immuni.2009.08.023 (2009).
33 Cipolletta, D., Cohen, P., Spiegelman, B. M., Benoist, C. & Mathis, D. Appearance and disappearance of the mRNA signature characteristic of Treg cells in visceral adipose tissue: age, diet, and PPARgamma effects. Proceedings of the National Academy of Sciences of the United States of America 112, 482-487, doi:10.1073/pnas.1423486112 (2015).
34 Bligh, E. G. & Dyer, W. J. A rapid method of total lipid extraction and purification. Canadian journal of biochemistry and physiology 37, 911-917, doi:10.1139/o59-099 (1959).
35 Lillington, J. M., Trafford, D. J. & Makin, H. L. A rapid and simple method for the esterification of fatty acids and steroid carboxylic acids prior to gas-liquid chromatography. Clinica chimica acta; international journal of clinical chemistry 111, 91-98 (1981).

1. A method of treating lymphedema in a patient in need thereof comprising administering to the patient a therapeutically effective amount of i) a 15-LOX polypeptide or ii) a polynucleotide encoding for a 15-LOX polypeptide.

2. The method of claim 1 wherein the 15-LOX polypeptide comprises an amino acid sequence having at least 90% of identity with the amino acid sequence that ranges from the amino acid residue at position 115 to the amino acid residue at position 662 in SEQ ID NO:1.

3. The method of claim 1 wherein the polynucleotide is a messenger RNA (mRNA).

4. The method of claim 1 wherein the polynucleotide is inserted in a vector.

5. The method of claim 4 wherein the viral vector is a AAV vector.

6. The method of claim 4 wherein the viral vector is a retroviral vector.

7. The method of claim 6 wherein the retroviral vector is a lentiviral vector.

8. A method of treating lymphedema in a patient in need thereof comprising administering to the patient a therapeutically effective amount of a 15-LOX metabolite, wherein the 15-LOX metabolite is 15-HETE.

9. The method of claim 4, wherein the vector is a viral vector.

10. The method of claim 9 wherein the viral vector is an adenoviral vector.

11. The method of claim 9 wherein the viral vector is a retroviral vector.

12. The method of claim 11 wherein the retroviral vector is a lentiviral vector.
